(19) United States
(12) Patent Application Publication
(10) Pub. No.: US 2023/0285468 A1
(43) Pub. Date: Sep. 14, 2023

(54) EFFECTS OF CELL-FREE FAT LIQUID EXTRACT ON MACROPHAGE POLARIZATION MODULATION AND DISEASE TREATMENT

(71) Applicant: SHANGHAI SEME CELL TECHNOLOGY CO., LTD., Shanghai (CN)

(72) Inventors: Bin WANG, Shanghai (CN); Li ZHAO, Shanghai (CN)

(21) Appl. No.: 18/001,260

(22) PCT Filed: Jun. 8, 2021
(86) PCT No.: PCT/CN2021/098944

§ 371 Date: Dec. 8, 2022

(30) Foreign Application Priority Data
Jun. 10, 2020 (CN) 202010525082.7

(51) Int. Cl. A61K 35/35; A61K 38/18; A61K 38/27; A61K 38/19; A61P 3/10

(52) U.S. Cl. CPC A61K 35/35; A61K 38/27; A61K 38/185; A61K 38/193; A61K 38/1808; A61K 38/1825; A61K 38/1833; A61K 38/1841; A61K 38/1858; A61K 38/1866; A61P 3/10

(57) ABSTRACT

The present invention involves effects of a cell-free fat liquid extract on macrophage polarization modulation and disease treatment. Specifically, provided by the present invention is a method of (i) promoting macrophage transformation from the M1 to the M2 subtype; (ii) preventing and/or treating diabetes and a complication thereof; (iii) preventing and/or treating inflammation; and/or (iv) improving insulin resistance. The cell-free fat extract of the present invention has uniquely advantageous effects in terms of promoting macrophage transformation from the M1 to the M2 subtype, prevention and/or treatment of diabetes, a complication thereof, and inflammation, and improving insulin resistance

### TECHNICAL FIELD

The invention relates to the field of drugs, in particular to the effects of cell-free fat liquid extract on macrophage polarization modulation and disease treatment.

### BACKGROUND

The immune inflammatory response plays a crucial role in the development of various pathological processes such as inflammatory diseases, metabolic diseases, infectious diseases, autoimmune diseases, and tissue damage repair, etc.. As an important member of the intrinsic immunity, macrophages influence and regulate the course of the immune inflammatory response through the secretion of cytokines and antigen presentation, etc. In recent years, more and more studies have shown that the dysregulation of macrophage number, distribution, function and polarization balance plays a critical or dominant role in many disease processes.

Macrophages in tissues originate from monocytes in blood and can migrate from peripheral blood into almost all tissues to participate in the regulation of tissue homeostasis and inflammatory response. They are functionally plastic and diverse, and can change significantly in function with different stimuli in the surrounding environment, called macrophage polarization, which can produce different phenotypes and functional subpopulations of macrophages, and can be broadly classified into classically activated macrophages (M1 type) and substitution-activated macrophages (M2 type) according to the function of activated macrophages. The M1 type has the function of secreting a large number of pro-inflammatory cytokines, such as TNF-a, IL-1β, IL-6, NO, and ROS/NOS products, Th1 chemokines, etc., which play an important role in the initiation of inflammatory response, causing apoptosis, tissue damage, and promoting foreign body clearance. In contrast, M2 macrophages play a very different role, suppressing the inflammatory response and promoting tissue damage repair in inflammatory diseases, secreting large amounts of anti-inflammatory cytokines such as IL-10 and TGF-β, etc., suppressing M1 macrophage-mediated inflammatory responses, promoting angiogenesis, tissue repair and wound healing, and promoting Th2 immunity. The M1 type or M2 type formed by macrophage polarization changes dynamically and transforms each other according to different signals in the environment, which can promote tissue regeneration and repair, promote inflammatory response, aggravate tissue damage, and the difference of their polarization subtypes will directly determine the outcome of inflammatory response. Therefore, abnormalities in macrophage polarization regulation/balance are key factors involved in the onset, development and regression of many inflammatory response-related diseases.

Therefore, there is a need in the field to develop a drug for the prevention and treatment of abnormalities in macrophage polarization regulation/balance and related diseases.

### SUMMARY OF THE INVENTION

The purpose of the present invention is to provide a use of a cell-free fat extract for promoting the macrophage transformation from the M1 to the M2 subtype, preventing and/or treating diabetes, a complication thereof, inflammation, and improving insulin resistance.

The first aspect of the present invention provides a use of cell-free fat extract in the manufacture of a composition or preparation for one or more uses selected from the group consisting of: (i) promoting macrophage transformation from M1 to M2 subtype; (ii) preventing and/or treating diabetes and a complication thereof; (iii) preventing and/or treating inflammation; and/or (iv) improving insulin resistance.

In another preferred embodiment, the diabetes is selected from the group consisting of type 1 diabetes, type 2 diabetes, and combinations thereof.

In another preferred embodiment, the diabetes comprises diabetes caused by insulin resistance.

In another preferred embodiment, the diabetes comprises obese diabetes.

In another preferred embodiment, the diabetes comprises diabetes caused by a high-fat diet.

In another preferred embodiment, the diabetic complication is selected from the group consisting of diabetic retinopathy, diabetes-associated uveitis, diabetic cataract, diabetic foot, diabetic cardiovascular complications, diabetic cerebrovascular disease, diabetic neuropathy, and combinations thereof.

In another preferred embodiment, the prevention and/or treatment of diabetes and complications thereof include prevention and/or treatment of one or more selected from the group consisting of:
(ii-1) lowering blood sugar levels;
(ii -2) improving insulin resistance
(ii -3) reducing macrophage infiltration in peripheral tissues.

In another preferred embodiment, the insulin resistance comprises obesity-induced insulin resistance.

In another preferred embodiment, the insulin resistance comprises insulin resistance caused by a high-fat diet.

In another preferred embodiment, the diabetes comprises insulin resistance caused by inflammation of peripheral tissues and organs and/or macrophage infiltration of peripheral tissues and organs.

In another preferred embodiment, the insulin resistance comprises insulin resistance caused by inflammation of peripheral tissues and organs and/or macrophage infiltration of peripheral tissues and organs.

In another preferred embodiment, the prevention and/or treatment of inflammation comprises reducing the levels of inflammatory factors.

In another preferred embodiment, the inflammation is obesity-associated inflammation.

In another preferred embodiment, the inflammatory factor is selected from the group consisting of IL-1b, IL-6, TNF-α, F4/80, and combinations thereof.

In another preferred embodiment, the inflammation comprises inflammation of peripheral tissues and organs.

In another preferred embodiment, the inflammation comprises obesity-induced inflammation. In another preferred embodiment, the inflammation comprises inflammation caused by a high-fat diet.

In another preferred embodiment, the improvement of insulin resistance includes one or more of the group consisting of:
(iv-1) improving inflammation of peripheral tissues and organs;
(iv-2) improving macrophage infiltration in peripheral tissues and organs.

In another preferred embodiment, the peripheral tissue is selected from the group consisting of fatty tissue, skeletal muscle tissue, and combinations thereof.

In another preferred embodiment, the fatty tissue comprises inguinal fatty tissue.

In another preferred embodiment, the skeletal muscle tissue comprises gastrocnemius tissue.

In another preferred embodiment, the organ comprises a liver.

In another preferred embodiment, the macrophages comprise macrophages expressing CD68.

In another preferred embodiment, the cell-free fat extract is a cell-free fat extract obtained from fat in human or non-human mammals.

In another preferred embodiment, the non-human mammal is a monkey, an orangutan, a cow, a pig, a dog, a sheep, a rat or a rabbit.

In another preferred embodiment, the composition or preparation comprises a pharmaceutical composition or preparation, a food composition or preparation, a health care composition or preparation, or a dietary supplement.

In another preferred embodiment, the composition or preparation further comprises a pharmaceutically, food, health product or a dietary acceptable carrier.

In another preferred embodiment, the dosage form of the composition or preparation is an oral preparation, an external preparation or an injection preparation.

In another preferred embodiment, the injection preparation is an intravenous injection preparation.

In another preferred embodiment, the composition or preparation is administered by topical, local, or subcutaneous injection.

In another preferred embodiment, the cell-free fat extract is free of cell and free of lipid droplets. In another preferred embodiment, the lipid droplets are oil droplets released after the fat cells are broken.

In another preferred embodiment, the “free of lipid droplets” means that the volume of oil droplets in the cell-free fat extract is less than 1%, preferably less than 0.5%, more preferably less than 0.1% in total liquid percentage.

In another preferred embodiment, the cells are selected from the group consisting of endothelial cells, adipose stem cells, macrophagocytic cells, stromal cells.

In another preferred embodiment, the “cell-free” means that the average number of cells in 1 ml of cell-free fat extract is ≤ 1, preferably ≤ 0.5, more preferably ≤ 0.1, or 0.

In another preferred embodiment, the cell-free fat extract is a naturally obtained nano-fat extract with additive-free.

In another preferred embodiment, the “additive-free” means that no solution, solvent, small molecule, chemical agent, and biological additive are added during the preparation of the fat extract except for the rinsing step.

In another preferred embodiment, the fat extract is obtained by centrifuging the fat tissue after emulsification.

In another preferred embodiment, the fat extract contains, but is not limited to, one or more components selected from the group consisting of: growth factors IGF-1, BDNF, GDNF, TGF-β, HGF, bFGF, VEGF, PDGF, EGF, NT-3, GH, G-CSF, and combinations thereof.

In another preferred example, the cell-free fat extract contains one or more components selected from the group consisting of: IGF-1, BDNF, GDNF, TGF-β, HGF, bFGF, VEGF, TGF-β1, HGF, PDGF, EGF, NT-3, GH, G-CSF, and combinations thereof.

In another preferred embodiment, the cell-free fat extract contains, but is not limited to, one or more components selected from the group consisting of IGF-1, BDNF, GDNF, bFGF, VEGF, TGF-β1, HGF, PDGF, and combinations thereof.

In another preferred embodiment, in the cell-free fat extract, the concentration of the IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml.

In another preferred embodiment, in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml.

In another preferred embodiment, in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml.

In another preferred embodiment, in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml.

In another preferred embodiment, in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml.

In another preferred embodiment, in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml.

In another preferred embodiment, in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml.

In another preferred embodiment, in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

In another preferred embodiment, the weight ratio of the IGF-1 to VEGF is 20-100:1, preferably 30-70:1, more preferably 40-60:1, and most preferably 45-55:1.

In another preferred embodiment, the weight ratio of BDNF to VEGF is 2-20:1, preferably 4-15:1, more preferably 6-12:1, and most preferably 8-9.5:1.

In another preferred embodiment, the weight ratio of GDNF to VEGF is 2-20: 1, preferably 4-15:1, more preferably 6-12:1, and most preferably 8.5-9.5:1.

In another preferred embodiment, the weight ratio of bFGF to VEGF is 0.2-8:1, preferably 0.5-5:1, more preferably 0.6-2:1, more preferably 0.8-1.6:1, and most preferably 1-1.5:1.

In another preferred embodiment, the weight ratio of TGF-β1 to VEGF is 1-20:1, preferably 1-15:1, more preferably 1-10:1, more preferably 2-8:1, more preferably 4-6:1.

In another preferred embodiment, the weight ratio of HGF to VEGF is 1-20:1, preferably 1-15:1, more preferably 1-10:1, more preferably 2-8:1, more preferably 4-5.5:1.

In another preferred embodiment, the weight ratio of PDGF to VEGF is 0.1-3: 1, preferably 0.2-2:1, more preferably 0.4-1.5:1, and most preferably 0.7-1.2:1.

In another preferred embodiment, the cell-free fat extract is a liquid.

In another preferred embodiment, the cell-free fat extract is prepared by the following method:
(1) providing an fatty tissue raw material, cutting the fatty tissue raw material and rinsing it (e. g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i. e. the fat layer containing fat cells);
(4) emulsifying the intermediate layer to obtain an emulsified fat mixture (also called nano-fat);
(5) centrifuging the emulsified fat mixture, thereby obtaining an intermediate liquid layer, i.e. a primary fat extract; and
(6) filtering and degerming the primary fat extract, thereby obtaining the cell-free fat extract.

The second aspect of the present invention provides a method for preparing cell-free fat extract, and the method comprises the steps of:
(1) providing an fatty tissue raw material, cutting the fatty tissue raw material and rinsing it (e. g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i. e. the fat layer containing fat cells);
(4) emulsifying the intermediate layer to obtain an emulsified fat mixture (also called nano-fat);
(5) centrifuging the emulsified fat mixture, thereby obtaining an intermediate liquid layer, i.e. a primary fat extract; and
(6) filtering and degerming the primary fat extract, thereby obtaining the cell-free fat extract.

In another preferred embodiment, in step (2), the centrifugation is performed at 800-2500 g, preferably 800-2000 g, more preferably 1000-1500 g, and most preferably 1100-1300 g.

In another preferred embodiment, in the step (2), the centrifugation time is 1-15 min, preferably 1-10 min, more preferably 1-8 min, and most preferably 1-5 min.

In another preferred embodiment, in the step (4), the emulsification is mechanical emulsification.

In another preferred embodiment, the mechanical emulsification is performed by repeated blowing by a syringe (e. g., blowing 20-200 times, preferably 20-150 times, more preferably 20-100 times, more preferably 30-50 times).

In another preferred embodiment, the blowing method is that two 10ml injection syringes are connected to a tee tube and repeatedly push at a constant speed.

In another preferred embodiment, in the step (4), the emulsification is by means of crushing through a tissue homogenizer.

In another preferred embodiment, the step (5) further includes freezing and thawing the emulsified fat mixture before the centrifugation treatment.

In another preferred embodiment, the thawed mixture is used for centrifugation after freezing and thawing treatment.

In another preferred embodiment, the freezing temperature is from -50° C. to -120° C., preferably from -60° C. to -100° C., more preferably from -70° C. to -90° C.

In another preferred embodiment, the thawing temperature is 20-40° C., preferably 25-40° C., more preferably 37° C.

In another preferred embodiment, the number of cycles of thawing after freezing is 1-5 (preferably 1, 2, 3 or 4).

In another preferred embodiment, in the step (5), after centrifugation, the emulsified fat mixture is layered into four layers, the first layer is an oil layer, the second layer is a residual fatty tissue layer, the third layer is a liquid layer (i. e., an intermediate liquid layer), and the fourth layer is a cell/tissue debris precipitation layer.

In another preferred embodiment, in step (5), the centrifugation is performed at 800-2500 g, preferably 800-2000 g, more preferably 1000-1500 g, and most preferably 1100-1300 g.

In another preferred embodiment, in the step (5), the centrifugation time is 1-15 min, preferably 1-10 min, more preferably 2-8 min, and most preferably 3-7 min.

In another preferred embodiment, in the step (5), the first layer, the second layer, the third layer and the fourth layer are sequentially arranged from top to bottom.

In another preferred embodiment, in the step (5), the intermediate liquid layer is a transparent or substantially transparent layer.

In another preferred embodiment, in the step (6), the filter pack is capable of removing fat cells from the primary fat extract.

In another preferred embodiment, in the step (6), the filtering and degerming are carried out through a filter (such as a 0.22 µm microporous filter membrane).

In another preferred embodiment, the filter is a microporous membrane filter.

In another preferred embodiment, the pore size of the microporous filter membrane is 0.05-0.8 µm, preferably 0.1-0.5 µm, more preferably 0.1-0.4 µm, more preferably 0.15-0.3 µm, more preferably 0.2-0.25 µm, and most preferably 0.22 µm.

In another preferred embodiment, in the step (6), the filtering and degerming is carried out by first filtering through a first filter that can filter cells, and then through a second filter(such as a 0.22 µm filter) that can filter pathogens (such as bacteria).

In another preferred embodiment, the step (6) further includes subpackaging the fat extract to form a subpackaging product. The subpacked extract can be stored at -20° C. for later use; it can be used directly after thawing at low temperature (e. g. -4° C.) or at normal temperature, or stored at low temperature (e. g. 4° C.) for a period of time for later use after thawing.

The third aspect of the present invention provides a cell-free fat extract, and the cell-free fat extract is obtained by the method of the second aspect of the present invention.

The fourth aspect of the present invention provides a composition or preparation, and the composition or preparation comprises (a) a cell-free fat extract of the third aspect of the present invention; and (b) a pharmaceutically, food, health care product or dietary acceptable carrier or excipient.

In another preferred embodiment, the dosage form of the composition or preparation is a powder, a granule, a capsule, an injection, a tincture, an oral liquid, a tablet or a lozenge.

In another preferred embodiment, the injectable agent is an intravenous or intramuscular injection.

In another preferred embodiment, the dosage form of the composition or preparation is a solid dosage form, a semi-solid dosage form, or a liquid dosage form, such as a solution, gel, cream, emulsion, ointments, cream, paste, cake, powder, patch, etc.

In another preferred embodiment, the percentage by mass of the cell-free fat extract in the composition or preparation is 5 wt%, preferably 1-20 wt%, based on the total weight of the cosmetic composition.

The fifth aspect of the present invention provides a method of preparing a composition or preparation according to the fourth aspect of the present invention, and the method comprises the step of: mixing the cell-free fat extract according to the third aspect of the present invention with a pharmaceutically, food, health care product or dietary acceptable carrier or excipient to form the composition or preparation.

The sixth aspect of the present invention provides a method of (i) promoting macrophages transformation from M1 to M2 subtype;(ii) preventing and/or treating diabetes and complications thereof; (iii) preventing and/or treating inflammation; and/or (iv) improving insulin resistance, comprising administering to a subject in need thereof a cell-free fat extract according to the third aspect of the present invention.

In another preferred embodiment, the subject is a human or non-human mammal.

In another preferred embodiment, the non-human mammal comprises a rodent, such as a rat, a mouse.

It should be understood that within the scope of the present invention, the above-mentioned technical features of the present invention and the technical features specifically described in the following (such as embodiments) can be combined with each other to form a new or preferred technical solution. Limited to space, it is not repeated here.

### DESCRIPTION OF THE DRAWINGS

FIG. 1 shows the number of positive cells and their percentages for the M1 subtype macrophage surface marker CD86 and the M2 subtype macrophage surface marker CD206 detected by flow cytometry.

FIG. 2 shows the results of glucose tolerance test.

FIG. 3 shows the results of insulin tolerance test.

FIG. 4 shows the relative expression of inflammatory factor genes in peripheral tissues and organs by RT-PCR.

FIG. 5 shows the results of macrophage marker CD68 staining in liver, fat and skeletal muscle tissues.

### DETAILED DESCRIPTION OF EMBODIMENTS

After extensive and in-depth research, the present inventors have developed for the first time a cell-free fat extract that effectively promotes macrophage transformation from the M1 to the M2 subtype, with excellent ameliorative effects on diabetes, inflammation and insulin resistance. On this basis, the present invention is completed.

### Terms

Unless otherwise defined, all technical and scientific terms used herein have the same meaning as generally understood by those skilled in the art to which the present invention belongs.

As used herein, the terms “include”, “contain” and “comprise” are used interchangeably, including not only open definitions, but also semi-closed, and closed definitions. In other words, the terms include “consisting of” and “consisting essentially”.

As used herein, “diabetes” refers to a metabolic disease characterized by hyperglycemia. Hyperglycemia is caused by defective secretion or impaired biological effects of insulin, or both. The long-term hyperglycemia in diabetes leads to chronic damage and dysfunction of various tissues, especially eyes, kidneys, heart, blood vessels, and nerves. Typically, the hyperglycemia includes type 1 diabetes and type 2 diabetes.

As used herein, “type 1 diabetes” can also be called insulin-dependent diabetes, is diabetes caused by an absolute deficiency of insulin in the body and occurs mostly in children and adolescents, but can also occur at all ages. The onset is relatively rapid, insulin in the body is absolutely insufficient, and ketosisacidosisis likely to occur, and must be treated with insulin to obtain satisfactory results, otherwise it will be life-threatening.

As used herein, “type 2 diabetes” refers to a condition in which the body’s ability to produce insulin is not completely lost, and in some patients, insulin is even produced in excess, but the effect of insulin is poor.

As used herein, “insulin resistance” is an abnormal physiological state in which the body’s response to endogenous secretion or exogenous injection of insulin decreases. Insulin resistance refers to the decrease in the efficiency of insulin promoting glucose uptake and utilization due to various reasons, and the body’s compensatory secretion of excessive insulin produces hyperinsulinemia to maintain the stability of blood sugar. Insulin resistance can easily lead to metabolic syndrome and type 2 diabetes. In the 50s, Yallow et al. applied the radioimmunoassay technique to determine plasma insulin concentration and found that patients with lower plasma insulin levels had higher insulin sensitivity, while those with higher plasma insulin were insensitive to insulin, thus introducing the concept of insulin resistance.

In the present invention, the term “prevention” means a method of preventing the onset of a disease and/or its attendant symptoms or protecting a subject from developing the disease. The “prevention” used herein also includes delaying the onset of the disease and/or its accompanying symptoms and reducing the risk of disease in the subject.

The “treatment” described in the present invention includes delaying and terminating the progression of the disease, or eliminating the disease, and does not require 100% inhibition, elimination and reversal. In some embodiments, the composition or pharmaceutical composition of the present invention reduces, inhibits and/or reverses diabetes, for example, by at least about 10%, at least about 30%, at least about 50%, or at least about 80%, compared to the level observed in the absence of the cell-free fat extract, composition, kit, food or health care kit, active ingredient combination described herein.

As used herein, “improvement” includes prevention, treatment, mitigation, reversal and alleviation, etc.

As used herein, “IL-1b” refers to interleukin-1b.

As used herein, “IL-6” refers to interleukin-6.

As used herein, “TNF-α” refers to tumor necrosis factor α.

As used herein, the term “IGF-1” is called insulin-like growth factors-1.

As used herein, the term “BDNF” is called brain-derived neurotrophic factor (BDNF).

As used herein, the term “GDNF” is called glial cell line-derived neurotrophic factor.

As used herein, the term “bFGF” is called basic fibroblast growth factor.

As used herein, the term “VEGF” is called vascular endothelial growth factor.

As used herein, the term “TGF-β1” is called transforming growth factor-β1.

As used herein, the term “HGF” is called hepatocyte growth factor.

As used herein, the term “PDGF” is called platelet derived growth factor.

As used herein, the term “EGF” is called Epidermal Growth Factor.

As used herein, the term “NT-3” is called neurotrophins-3.

As used herein, the term “GH” is called Growth Hormone .

As used herein, the term “G-CSF” is called granulocyte colony stimulating factor.

### Cell Free Fat Extract (CEFFE) and Preparation Method Thereof

As used herein, the terms “cell-free fat extract of the present invention”, “extract of the present invention”, “fat extract of the present invention” and the like are used interchangeably to refer to an extract (or extract liquid) derived from fatty tissue prepared without adding any solutions, solvents, small molecules, chemicals, and biological additives during the preparation of the fat extract (other than the rinsing step). A typical process for preparing an extract of the present invention is as described above in the second aspect of the present invention. In addition, it should be understood that although the extract of the present invention does not need to add any additives (or additive ingredients) during the preparation process, some or a small amount of a safe substance (such as a small amount of water) that does not negatively or adversely affect the activity of the extract of the present invention can also be added.

The cell-free fat extract of the present invention can be derived from human fatty tissue, which is purified from nano-fat by removing oil and cell/extracellular matrix parts after centrifugation, and is a cell-free, easy-to-prepare liquid, and rich in various growth factors.

In a preferred embodiment of the present invention, the cell-free fat extract is a cell-free fat extract liquid.

The cell-free fat extract described in the present invention may include a variety of cytokines. Typically, the cell-free fat extract comprises one or more of IGF-1, BDNF, GDNF, TGF-β, HGF, bFGF, VEGF, TGF-β1, PDGF, EGF, NT-3, GH, and G-CSF.

In another preferred embodiment, the cell-free fat extract contains, but is not limited to, one or more components selected from the group consisting of IGF-1, BDNF, GDNF, bFGF, VEGF, TGF-β1, HGF, PDGF, and combinations thereof.

In another preferred embodiment, in the cell-free fat extract, the concentration of the IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml.

In another preferred embodiment, in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml.

In another preferred embodiment, in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml.

In another preferred embodiment, in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml.

In another preferred embodiment, in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml.

In another preferred embodiment, in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml.

In another preferred embodiment, in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml.

In another preferred embodiment, in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

In another preferred embodiment, the weight ratio of the IGF-1 to VEGF is 20-100:1, preferably 30-70:1, more preferably 40-60:1, and most preferably 45-55:1.

In another preferred embodiment, the weight ratio of BDNF to VEGF is 2-20:1, preferably 4-15:1, more preferably 6-12:1, and most preferably 8-9.5:1.

In another preferred embodiment, the weight ratio of GDNF to VEGF is 2-20: 1, preferably 4-15:1, more preferably 6-12:1, and most preferably 8.5-9.5:1.

In another preferred embodiment, the weight ratio of bFGF to VEGF is 0.2-8:1, preferably 0.5-5:1, more preferably 0.6-2:1, more preferably 0.8-1.6:1, and most preferably 1-1.5:1.

In another preferred embodiment, the weight ratio of TGF-β1 to VEGF is 1-20:1, preferably 1-15:1, more preferably 1-10:1, more preferably 2-8:1, more preferably 4-6:1.

In another preferred embodiment, the weight ratio of HGF to VEGF is 1-20:1, preferably 1-15:1, more preferably 1-10:1, more preferably 2-8:1, more preferably 4-5.5:1.

In another preferred embodiment, the weight ratio of PDGF to VEGF is 0.1-3: 1, preferably 0.2-2:1, more preferably 0.4-1.5:1, and most preferably 0.7-1.2:1.

Preferably, the cell-free fat extract of the present invention is obtained by the method of the second aspect of the present invention.

Typically, the cell-free fat extract described in the present invention is prepared by the following methods:
(1) providing an fatty tissue raw material, cutting the fatty tissue raw material and rinsing it (e. g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i. e. the fat layer containing fat cells);
(4) emulsifying the intermediate layer to obtain an emulsified fat mixture (also called nano-fat);
(5) centrifuging the emulsified fat mixture, thereby obtaining an intermediate liquid layer, i.e. a primary fat extract; and
(6) filtering and degerming the primary fat extract, thereby obtaining the cell-free fat extract.

In another preferred embodiment, in step (2), the centrifugation is performed at 800-2500 g, preferably 800-2000 g, more preferably 1000-1500 g, and most preferably 1100-1300 g.

In another preferred embodiment, in the step (2), the centrifugation time is 1-15 min, preferably 1-10 min, more preferably 1-8 min, and most preferably 1-5 min.

In another preferred embodiment, in the step (4), the emulsification is mechanical emulsification.

In another preferred embodiment, the mechanical emulsification is performed by repeated blowing by a syringe (e. g., blowing 20-200 times, preferably 20-150 times, more preferably 20-100 times, more preferably 30-50 times).

In another preferred embodiment, the blowing method is that two 10ml injection syringes are connected to a tee tube and repeatedly push at a constant speed.

In another preferred embodiment, in the step (4), the emulsification is by means of crushing through a tissue homogenizer.

In another preferred embodiment, the step (5) further includes freezing and thawing the emulsified fat mixture before the centrifugation treatment.

In another preferred embodiment, the thawed mixture is used for centrifugation after freezing and thawing treatment.

In another preferred embodiment, the freezing temperature is from -50° C. to -120° C., preferably from -60° C. to -100° C., more preferably from -70° C. to -90° C.

In another preferred embodiment, the thawing temperature is 20-40° C., preferably 25-40° C., more preferably 37° C.

In another preferred embodiment, the number of cycles of thawing after freezing is 1-5 (preferably 1, 2, 3 or 4).

In another preferred embodiment, in the step (5), after centrifugation, the emulsified fat mixture is layered into four layers, the first layer is an oil layer, the second layer is a residual fatty tissue layer, the third layer is a liquid layer (i. e., an intermediate liquid layer), and the fourth layer is a cell/tissue debris precipitation layer.

In another preferred embodiment, in step (5), the centrifugation is performed at 800-2500 g, preferably 800-2000 g, more preferably 1000-1500 g, and most preferably 1100-1300 g.

In another preferred embodiment, in the step (5), the centrifugation time is 1-15 min, preferably 1-10 min, more preferably 2-8 min, and most preferably 3-7 min.

In another preferred embodiment, in the step (5), the first layer, the second layer, the third layer and the fourth layer are sequentially arranged from top to bottom.

In another preferred embodiment, in the step (5), the intermediate liquid layer is a transparent or substantially transparent layer.

In another preferred embodiment, in the step (6), the filter pack is capable of removing fat cells from the primary fat extract.

In another preferred embodiment, in the step (6), the filtering and degerming are carried out through a filter (such as a 0.22 µm microporous filter membrane).

In another preferred embodiment, the filter is a microporous membrane filter.

In another preferred embodiment, the pore size of the microporous filter membrane is 0.05-0.8 µm, preferably 0.1-0.5 µm, more preferably 0.1-0.4 µm, more preferably 0.15-0.3 µm, more preferably 0.2-0.25 µm, and most preferably 0.22 µm.

In another preferred embodiment, in the step (6), the filtering and degerming is carried out by first filtering through a first filter that can filter cells, and then through a second filter(such as a 0.22 µm filter) that can filter pathogens (such as bacteria).

In another preferred embodiment, the step (6) further includes subpackaging the fat extract to form a subpackaging product. The subpacked extract can be stored at -20° C. for later use; it can be used directly after thawing at low temperature (e. g. -4° C.) or at normal temperature, or stored at low temperature (e. g. 4° C.) for a period of time for later use after thawing.

### Use

The cell-free fat extract described in the present invention can effectively promote macrophages transformation from M1 to M2 subtype, and has excellent improvement effect on diabetes, inflammation and insulin resistance.

Typically, the cell-free fat extract of the present invention comprises one or more uses selected from the group consisting of: (i) promoting macrophage transformation from the M1 to the M2 subtype; (ii) preventing and/or treating diabetes and a complication thereof; (iii) preventing and/or treating inflammation; and/or (iv) improving insulin resistance.

In another preferred embodiment, the diabetes is selected from the group consisting of type 1 diabetes, type 2 diabetes, and combinations thereof.

In another preferred embodiment, the diabetes comprises diabetes caused by insulin resistance. In another preferred embodiment, the diabetes comprises obese diabetes.

In another preferred embodiment, the diabetes comprises diabetes caused by a high-fat diet. Typically, the diabetic complication is selected from the group consisting of diabetic retinopathy, diabetes-associated uveitis, diabetic cataract, diabetic foot, diabetic cardiovascular complications, diabetic cerebrovascular disease, diabetic neuropathy, and combinations thereof.

In another preferred embodiment, the prevention and/or treatment of diabetes and complications thereof include prevention and/or treatment of one or more selected from the group consisting of:
(ii-1) lowering blood sugar levels;
(ii -2) improving insulin resistance
(ii -3) reducing macrophage infiltration in peripheral tissues.

In another preferred embodiment, the insulin resistance comprises obesity-induced insulin resistance.

In another preferred embodiment, the insulin resistance comprises insulin resistance caused by a high-fat diet.

In another preferred embodiment, the diabetes comprises insulin resistance caused by inflammation of peripheral tissues and organs and/or macrophage infiltration of peripheral tissues and organs.

In another preferred embodiment, the insulin resistance comprises insulin resistance caused by inflammation of peripheral tissues and organs and/or macrophage infiltration of peripheral tissues and organs.

In another preferred embodiment, the prevention and/or treatment of inflammation comprises reducing the levels of inflammatory factors.

In another preferred embodiment, the inflammatory factor is selected from the group consisting of IL-1b, IL-6, TNF-α, F4/80, and combinations thereof.

In another preferred embodiment, the inflammation comprises inflammation of peripheral tissues and organs.

In another preferred embodiment, the inflammation comprises obesity-induced inflammation.

In another preferred embodiment, the inflammation comprises inflammation caused by a high-fat diet.

In another preferred embodiment, the improvement of insulin resistance includes one or more of the group consisting of:
(iv-1) improving inflammation of peripheral tissues and organs;
(iv-2) improving macrophage infiltration in peripheral tissues and organs.

In another preferred embodiment, the peripheral tissue is selected from the group consisting of fatty tissue, skeletal muscle tissue, and combinations thereof.

In another preferred embodiment, the fatty tissue comprises inguinal fatty tissue.

In another preferred embodiment, the skeletal muscle tissue comprises gastrocnemius tissue.

In another preferred embodiment, the organ comprises a liver.

In another preferred embodiment, the macrophages comprise macrophages expressing CD68.

The present invention also provides a method of (i) promoting macrophages transformation from M1 to M2 subtype; (ii) preventing and/or treating diabetes and complications thereof; (iii) preventing and/or treating inflammation; and/or (iv) improving insulin resistance, comprising administering to a subject in need thereof a cell-free fat extract of the present invention.

In another preferred embodiment, the subject is a human or non-human mammal.

In another preferred embodiment, the non-human mammal comprises a rodent, such as a rat, a mouse.

### Composition and Application

The compositions described herein include, but are not limited to, pharmaceutical compositions, food compositions, health-care compositions, dietary supplements, and the like.

Typically, the cell-free fat extract of the present invention can be prepared as pharmaceutical compositions in dosage forms such as tablets, capsules, powders, microgranule, solutions, lozenges, jellies, cream, spiritus, suspensions, tinctures, mud dressings, liniment, lotions, and aerosols, etc. Pharmaceutical compositions can be prepared by commonly known preparation techniques, and suitable pharmaceutical additives can be added to the drug.

The compositions of the present invention can also include pharmaceutically, food, health care product or dietary acceptable carriers. “Pharmaceutically, food, health care product or dietary acceptable carriers” means one or more compatible solid or liquid filler or gel substances that are suitable for human use and must have sufficient purity and sufficiently low toxicity. “Compatibility” herein refers to the ability of components of a composition to blend with the compounds of the invention and with each without significantly reducing the efficacy of the compounds. Examples of pharmaceutically, food, health care product or dietary acceptable carriers include cellulose and its derivatives (such as sodium carboxymethyl cellulose, sodium ethyl cellulose, cellulose acetate, etc.), gelatin, talc, solid lubricants (such as stearic acid, Magnesium stearate), calcium sulfate, vegetable oil (such as soybean oil, sesame oil, peanut oil, olive oil, etc.), polyols (such as propylene glycol, glycerin, mannitol, sorbitol, etc.), emulsifier (such as Tween®), wetting agents (such as sodium dodecyl sulfate), colorants, flavoring agents, stabilizers, antioxidants, preservatives, pyrogen-free water, etc.

The methods of administration of the compositions of the present invention are not particularly limited, and representative methods of administration include, but are not limited to, oral, parenteral (intravenous, intramuscular), topical administration, preferably oral administration and injection administration.

Solid dosage forms for oral administration include capsules, tablets, pills, powders and granules. In these solid dosage forms, the active compounds is mixed with at least one conventional inert excipient (or carrier), such as sodium citrate or dicalcium phosphate, or mixed with:(a) fillers or compatibilizers, e.g., starch, lactose, sucrose, glucose, mannitol and silicic acid; (b) binders, e.g., hydroxymethylcellulose, alginate, gelatin, polyvinylpyrrolidone, sucrose, and gum arabic; (c) humectants, e.g., glycerol; (d) disintegrants, e.g., agar, calcium carbonate, potato starch or tapioca starch, alginic acid, certain complex silicates, and sodium carbonate; (e) dissolution-retarding agents, e.g., paraffin; (f) absorption accelerators, e.g., quaternary amine compounds; (g) wetting agents, e.g., cetearyl alcohol and glycerol monostearate; (h) sorbents, e.g., kaolin; and (i) lubricants, e.g., talc, calcium stearate, magnesium stearate, solid polyethylene glycol, sodium dodecyl sulfate, or mixtures thereof. In capsules, tablets and pills, dosage forms may also contain buffers.

Solid dosage forms such as tablets, sugar pills, capsules, pilula and granules may be prepared using coating and shell materials such as casing and other materials well known in the art. They can contain opaque agents.

Liquid dosage forms for oral administration include pharmaceutically acceptable emulsions, solutions, suspensions, syrups or tinctures. In addition to the active compounds, the liquid dosage form may contain inert diluents conventionally used in the art, such as water or other solvents, solubilizers and emulsifiers, for example, ethanol, isopropanol, ethyl carbonate, ethyl acetate, propylene glycol, 1,3-butanediol, dimethylformamide and oils, especially cottonseed oil, peanut oil, corn germ oil, olive oil, castor oil and sesame oil, or mixtures thereof.

In addition to these inert diluents, the composition may also contain auxiliaries such as wetting agents, emulsifiers, suspending agents, sweeteners, flavoring agents and flavors.

In addition to the active ingredient, the suspension may comprise suspending agents, such as ethoxylated isooctadecanol, polyoxyethylene sorbitol and dehydrated sorbitol esters, microcrystalline cellulose, methanolic aluminum, agar, and any mixtures thereof.

The composition for parenteral injection may comprise physiologically acceptable sterile aqueous or anhydrous solutions, dispersions, suspensions or emulsions, and sterile powders for redissolution into sterile injectable solutions or dispersions. Suitable aqueous and non-aqueous carriers, diluents, solvents, or excipients include water, ethanol, polyols, and suitable mixtures thereof.

Dosage forms of the compounds of the invention for topical administration include ointments, powder, patches, sprays and inhalants. The active ingredient is mixed under sterile conditions with a physiologically acceptable carrier and any preservatives buffers or propellants as may be required.

The cell-free fat extract of the present invention can be administered alone, or in combination with other drugs for the prevention and/or treatment of fatty liver and/or its complications.

When the composition is administered, a safe and effective amount of the cell-free fat extract of the present invention is applied to a human or non-human animal in need of treatment (e. g., rat, mouse, dog, cat, cow, chicken, duck, etc.) at a dose that is pharmaceutically, food or dietary acceptable to the effective administration. As used herein, the term “safe and effective amount” refers to an amount that produces function or activity to humans and/or animals and is acceptable to humans and/or animals. Those ordinary skille’d in the art will understand that the “safe and effective amount” described may vary depending on the form of the pharmaceutical composition, the route of administration, the excipient of the drug used, the severity of the disease, and the combination with other drugs. For example, for a person of 60 kg body weight, the daily dose is usually 0.1 to 1000 mg, preferably 1 to 600 mg, more preferably 2 to 300 mg. Of course, the specific dosage should also consider the route of administration, the patient’s health and other factors, which are within the skill range of skilled doctors.

### The Main Advantages of the Present Invention Include

The present invention found for the first time that cell-free fat extract can promote the transformation of macrophages from M1 to M2 subtype, and uses high-fat diet-induced type II diabetes model mice as a model, confirmed that CEFFE treatment can regulate peripheral tissue macrophage polarization, reduce macrophage recruitment, improve obesity-related chronic inflammation, and enhance insulin sensitivity, which suggests the therapeutic potential of CEFFE in obesity-related metabolic diseases, and likewise suggests the therapeutic value of CEFFE in diseases associated with immune inflammatory responses involving macrophage polarization.

The present invention is further described below in conjunction with specific examples. It is to be understood that these examples are intended to illustrate the invention only and not to limit the scope of the invention. The following examples do not indicate the specific conditions of the experimental method, usually according to the conventional conditions, or according to the conditions recommended by the manufacturer. Percentages and parts are calculated by weight unless otherwise stated.

### Example 1

### 1. Preparation of Cell Free Fat Extract (CEFFE)

Fat is obtained by volunteers with informed consent. The preparation method of cell free fat tissue extract is as follows:

The fat obtained by aspiration or surgical excision was taken, cut up and rinsed 3 times with normal saline.

The rinsed fatty tissue was taken, placed in a centrifuge tube, then placed in a centrifuge, and centrifuged at 1200 g for 3 minutes to obtain a layered mixture.

For the layered mixture, the upper oil layer and the lower water layer were removed and the intermediate layer (i.e. the fat layer containing fat cells) was collected.

For the intermediate layer, two 10 ml syringes connected to a tee tube were pushed repeatedly and uniformly for 30 times, thus performing mechanical emulsification and obtaining a mechanically emulsified fat mixture (also called nano-fat).

The mechanically emulsified fat mixture was placed into a -80° C. refrigerator for freezing, and then thawed in a 37° C. water bath, and after a single freeze-thaw cycle, the thawed fat mixture was centrifuged at 1200 g for 5 minutes to obtain a layered mixture, which was divided into 4 layers, the first layer being the oil layer, the second layer being the residual fatty tissue layer, the third layer being the liquid layer, and the fourth layer being the cell/tissue debris precipitation layer, the oil layer and the residual fatty tissue layer were removed and the liquid layer was aspirated, avoiding contamination of the cellular/tissue debris precipitation layer during the aspiration process, resulting in a primary fat extract.

The obtained primary fat extract was filtered and degermed through a 0.22 µm filter, thereby sterilizing and removing any live cells that may have been mixed, resulting in a cell-free fat extract that was subpackaged and stored frozen at -20° C. and thawed at 4° C. when used.

The content of cytokines, including IGF-1, BDNF, GDNF, bFGF, VEGF, TGF-β1, HGF and PDGF of the obtained cell-free fat extract, were detected by ELISA immunosorbent assay kit. The average concentrations of 6 samples were as follows: IGF-1 (9840.6 pg/ml), BDNF (1764.5 pg/ml), GDNF (1831.9 pg/ml), bFGF (242.3 pg/ml), VEGF (202.9 pg/ml), TGF-β1 (954.5 pg/ml), HGF (898.4 pg/ml), PDGF (179.9 pg/ml).

### 2. Cell Culture

The mouse monocyte macrophage cell line RAW264.7(M0 macrophage) was purchased from the Chinese Academy of Sciences cell bank, and lipopolysaccharide (LPS) and interferon-y(IFN-γ) were purchased from Sigma Company of the United States. RAW264.7(M0 macrophage) was cultured in a 5% CO2 incubator at 37° C. with high glucose DMEM with 10% fetal bovine serum medium. The medium was changed every other day and passaged at 90% density to obtain M0 macrophage culture.

Macrophage culture medium was added with 100 ng/mL LPS and 30 ng/mL IFN to establish an in vitro inflammation model.

CEFFE-treated group was added to the cell culture medium with 10% concentration of CEFFE (v/v).

### 3. Flow Cytometry Assay

The M0 macrophages cultured in vitro in step 2 were divided into blank group (Control group), CEFFE group, LPS + IFN-γ group and LPS + IFN-γ + CEFFE group; in the CEFFE group, CEFFE was added to the M0 macrophage culture medium at a concentration of 10%(v/v) of cell culture medium; in the LPS + IFN-γ group, LPS(100 ng/mL) and IFN-γ(30 ng/mL) were added to M0 macrophage culture medium; in LPS + IFN-γ + CEFFE group, LPS(100 ng/mL), IFN-γ(30 ng/mL) and CEFFE (at a concentration of 10%(v/v) cell culture medium) were added to M0 macrophage culture medium; no drug was added to the Control group. After different groups of M0 macrophages were cultured in vitro for 24 h, the cells were digested, collected, incubated with fluorescently labeled CD86 and CD206 antibodies, respectively, at 4° C. for 30 min, washed, resuspended, and detected by flow cytometry for cell fluorescence expression.

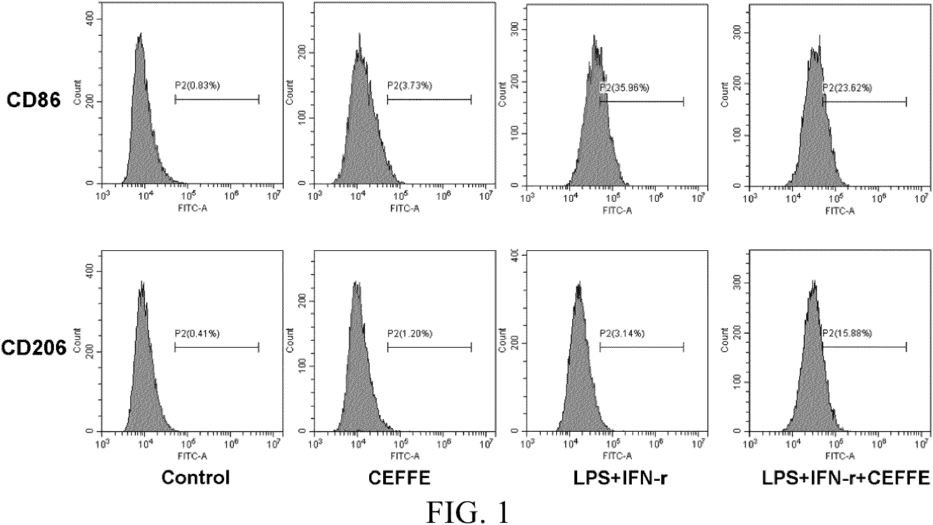

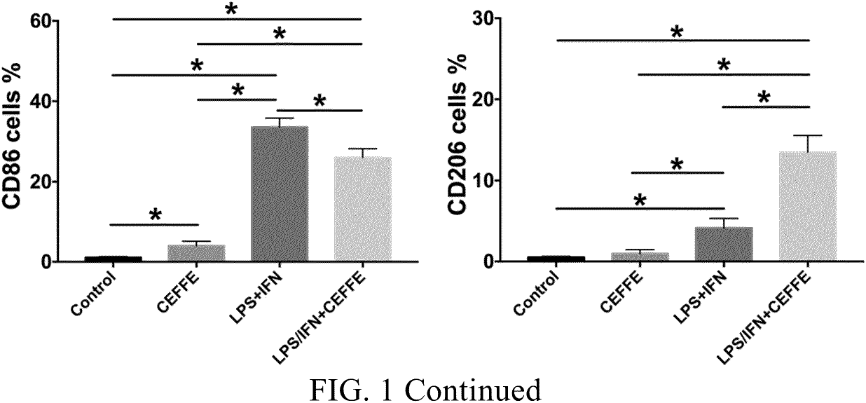

LPS and IFN were able to stimulate the polarization of M0 macrophages towards M1 subtype, thus establishing an in vitro model of inflammation. CD86 is the M1 subtype macrophage surface marker and CD206 is the M2 subtype macrophage surface marker, and the number of positive cells and their percentage of M1 subtype macrophage surface marker CD86 and M2 subtype macrophage surface marker CD206 were detected by flow cytometry after 24 h of culture as shown in FIG. 1.

As can be seen in FIG. 1, compared with the Control group, CEFFE culture in the CEFFE group failed to result in a significant polarized subtype of M0 macrophages, and although the percentage of CD86+ cells increased slightly to 3.73%, it was still much lower than that of the LPS+ IFN-γ group (35.96%). Compared with the M1 conventionally induced LPS+ IFN-γ group, the addition of CEFFE was able to significantly decrease the percentage of CD86+ cells and increase the percentage of CD206+ cells in the LPS+ IFN-γ+CEFFE group, and the decrease and increase in the percentage of cells of both phenotypes were essentially the same (about 12%), indicating that CEFFE treatment promoted the macrophage subtype from M1 to M2 transformation.

### 4. Establishment of High-Fat-Induced Insulin Resistance Mouse Model and Pharmacodynamic Study

### 4.1 Establishment, Grouping and Drug Administration of High-Fat-Induced Insulin Resistance Mouse Model

The 6-week-old C57 mice were purchased from Shanghai Experimental Animal Center, and the mouse insulin resistance model was induced by high-fat diet for 15 consecutive weeks. After 15 weeks of feeding, the mice were tested for fasting glucose tolerance and insulin tolerance. Subsequently, the mice were divided into three groups: blank control group (normal diet, Chow group), negative control group (high-fat diet + injection of PBS, PBS group) and CEFFE group (high-fat diet + injection of CEFFE, CEFFE group). The tail vein was injected once in 4 days for a total of 7 injections in a 30-day treatment cycle,, in which each CEFFE injection dose was 250 µl, with PBS as a negative control, CHOW as a blank control. Randomized blood glucose and body weight were regularly monitored during treatment.

After 30 days of treatment, fasting glucose tolerance test and insulin tolerance test were performed again, and blood was drawn from mice for hematological examination; liver, inguinal fat and gastrocnemius muscle tissues of model mice were taken and RT-PCR was performed to detect the expression of inflammatory factors in peripheral tissues and immunostaining, respectively, and the results were as follows:

### 4.2 High-Fat-Induced Insulin Resistance Mouse Model for Pharmacodynamic Study

Statistical analysis: the data were statistically analyzed by one-way ANOVA test using SPSS software for the significance of data differences, and the results were expressed as mean ± standard deviation.

### 4.2.1 Glucose Tolerance and Insulin Tolerance Test

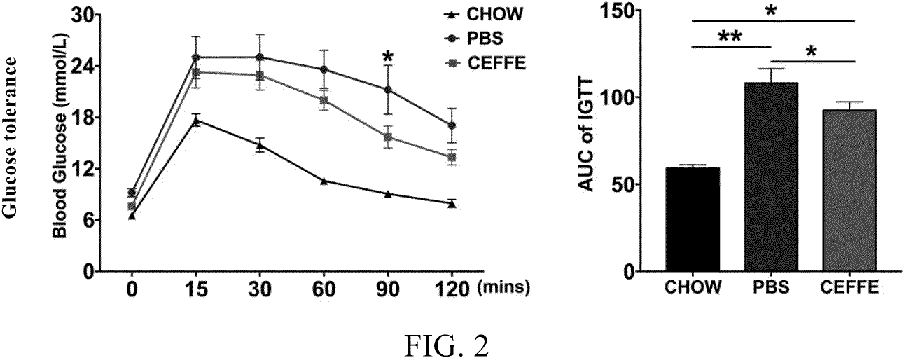

For glucose tolerance test: glucose were injected intraperitoneally after 12 h fasting, and the tail vein blood glucose levels were measured at 0, 15, 30, 60, 90 and 120 min, respectively. Glucose tolerance test is shown in FIG. 2.

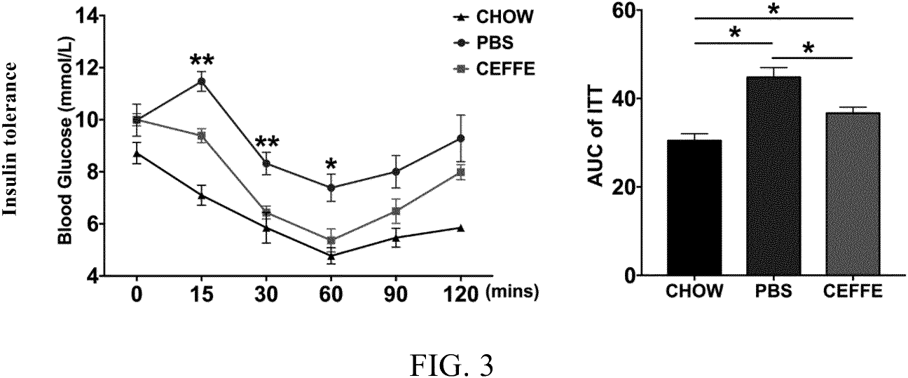

For insulin tolerance test: mice were injected with insulin intraperitoneally after 6 h fasting, and the tail vein blood glucose levels were measured at 0, 15, 30, 60, 90 and 120 min, respectively. The insulin tolerance test is shown in FIG. 3.

As can be seen from FIGS. 2 and 3, compared with the blank control group, the blood glucose level of the mice in the high-fat diet negative control group at 0, 15, 30, 60, 90, 120 min was significantly increased, and the area under the glucose tolerance and insulin tolerance curve (AUC) was significantly increased. Compared with the negative control group, the blood glucose level and AUC in CEFFE treatment group were significantly reduced, indicating that CEFFE treatment can improve insulin sensitivity and insulin resistance. The above results show that CEFFE treatment can improve insulin resistance by regulating macrophage polarization in peripheral tissues.

### 4.2.2 Detection of Inflammatory Factor Expression in Peripheral Tissues by RT-PCR

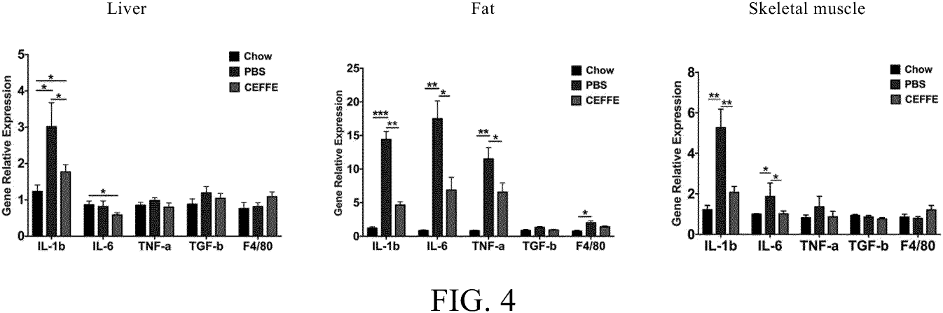

Liver, inguinal fat and gastrocnemius muscle tissues of model mice were taken, total RNA was extracted by Trizol, and RNA concentration was calculated by spectrophotometric method at 260/280 nm. After reverse transcription with EZbioscience kit, the expression levels of IL-1b, IL-6, TNF-a and F4/80 were detected by RT-PCR fluorescence. The total reaction system was 20 ul, and the amplification conditions were: initial denaturation at 95° C. for 10 min; 15 s at 95° C., 60s at 62° C., 40 cycles. Relative quantitative statistics of RT-PCR results were performed at the end of the reaction, and the relative expression of inflammatory factor genes in peripheral tissues and organs detected by RT-PCR is shown in FIG. 4.

As can be seen from FIG. 4, compared with the blank control group, the expression of inflammatory factors in liver, fat and skeletal muscle tissues was increased in the high-fat fed negative control mice, while it was significantly decreased after CEFFE treatment, indicating that CEFFE can effectively reduce peripheral tissue and organ inflammation in diabetic mice.

### 4.2.3 Immunostaining

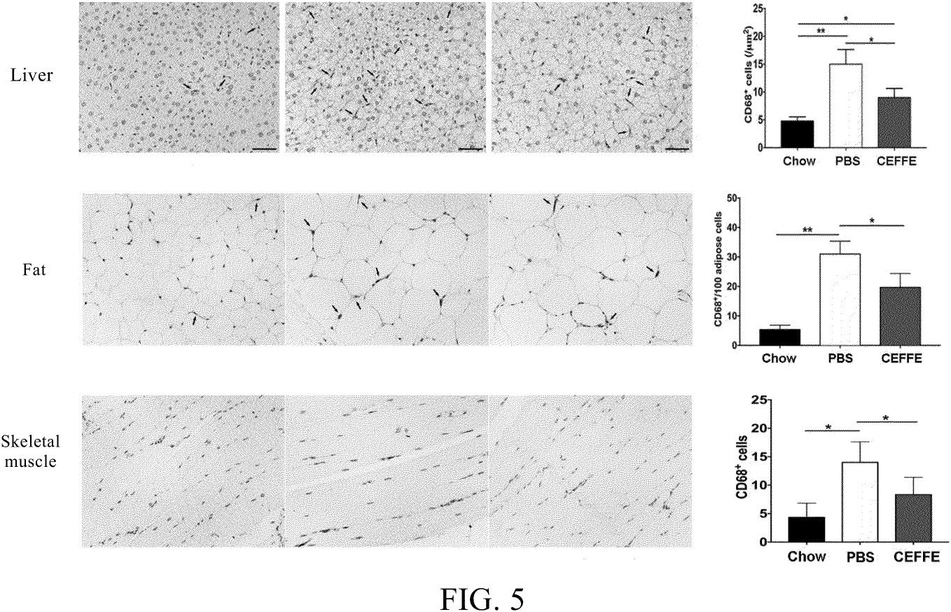

The liver, inguinal fat and gastrocnemius muscle tissues of model mice were taken, dipped in 4% paraformaldehyde, fixed for 24 h and then paraffin-embedded, routinely done in paraffin sections, dewaxed, hydrated and then repaired under high pressure, blocked with 5% BSA and then incubated with 1:100 CD68 overnight at 4° C., the next day after full washing and dropwise addition of HRP-secondary antibody, incubated at 37° C. for 30 min and followed by DAB color development. Optical microscope photographs were taken and ImageJ counting analysis was performed, and the macrophage marker CD68 staining in liver, fat and skeletal muscle tissues is shown in FIG. 5.

As can be seen from FIG. 5, the number of CD68+ macrophages was significantly decreased in the CEFFE-treated group compared with the negative control group, indicating that CEFFE treatment could effectively reduce macrophage infiltration in peripheral tissues.

All documents referred to in the present invention are incorporated by reference herein as if each document is individually incorporated by reference. Further, it should be understood that upon reading the above teaching of the present invention, various variations or modifications may be made to the present invention by those skilled in the art, and those equivalents also fall within the scope defined by the appended claims of the present application.

1. A method of (i) promoting macrophage transformation from M1 to M2 subtype; (ii) preventing and/or treating diabetes and a complication thereof; (iii) preventing and/or treating inflammation; and/or (iv) improving insulin resistance, comprising administering an effective amount of a composition to a subject in need thereof, wherein the composition comprises a cell-free fat extract.

2. The method of claim 1, wherein the diabetes is selected from the group consisting of type 1 diabetes, type 2 diabetes, and combinations thereof.

3. The method of claim 1, wherein the diabetes comprises diabetes caused by insulin resistance.

4. The method of claim 1, wherein the diabetes comprises diabetes caused by a high-fat diet.

5. The method of claim 1, wherein the prevention and/or treatment of diabetes and complications thereof comprises prevention and/or treatment of one or more selected from the group consisting of:
(ii-1) lowering blood sugar levels;
(ii -2) improving insulin resistance;
(ii -3) reducing macrophage infiltration in peripheral tissues.

6. The method of claim 1, wherein the insulin resistance comprises insulin resistance caused by a high-fat diet.

7. The method of claim 1, wherein the insulin resistance comprises insulin resistance caused by inflammation of peripheral tissues and organs and/or macrophage infiltration of peripheral tissues and organs.

8. The method of claim 1, wherein the improving insulin resistance comprises improving one or more selected from the group consisting of:
(iv-1) improving inflammation of peripheral tissues and organs;
(iv-2) improving macrophage infiltration in peripheral tissues and organs.

9. The method of claim 1, wherein the cell-free fat extract is prepared by the following method:
(1) providing an fatty tissue raw material, cutting the fatty tissue raw material and rinsing it (e. g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i. e. the fat layer containing fat cells);
(4) emulsifying the intermediate layer to obtain an emulsified fat mixture (also called nano-fat);
(5) centrifuging the emulsified fat mixture, thereby obtaining an intermediate liquid layer, i.e. a primary fat extract; and
(6) filtering and degerming the primary fat extract, thereby obtaining the cell-free fat extract.

10. The method of claim 1, wherein the cell-free fat extract comprises one or more components selected from the group consisting of IGF-1, BDNF, GDNF, HGF, bFGF, VEGF, TGF-β1, HGF, PDGF, EGF, NT-3, GH, G-CSF, and combinations thereof.

11. The method of claim 10, wherein the cell-free fat extract comprises one or more features selected from the group consisting of:
in the cell-free fat extract, the concentration of the IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml;
in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml;
in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml;
in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml;
in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml;
in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml;
in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 p g/ml; and/or
in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

12. The method of claim 10, wherein the cell-free fat extract comprises one or more features selected from the group consisting of:
the weight ratio of the IGF-1 to VEGF is 20-100:1, preferably 30-70:1, more preferably 40-60:1, and most preferably 45-55:1;
the weight ratio of BDNF to VEGF is 2-20:1, preferably 4-15:1, more preferably 6-12:1, and most preferably 8-9.5:1;
the weight ratio of GDNF to VEGF is 2-20:1, preferably 4-15:1, more preferably 6-12:1, and most preferably 8.5-9.5:1;
the weight ratio of bFGF to VEGF is 0.2-8:1, preferably 0.5-5:1, more preferably 0.6-2:1, more preferably 0.8-1.6:1, and most preferably 1-1.5:1;
the weight ratio of TGF-β1 to VEGF is 1-20:1, preferably 1-15:1, more preferably 1-10:1, more preferably 2-8:1, more preferably 4-6:1;
the weight ratio of HGF to VEGF is 1-20:1, preferably 1-15:1, more preferably 1-10:1, more preferably 2-8:1, more preferably 4-5.5:1; and/or
the weight ratio of PDGF to VEGF is 0.1-3:1, preferably 0.2-2:1, more preferably 0.4-1.5:1, and most preferably 0.7-1.2:1.

13. A method for preparing cell-free fat extract, wherein the method comprises the steps of:
(1) providing an fatty tissue raw material, cutting the fatty tissue raw material and rinsing it (e. g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i. e. the fat layer containing fat cells);
(4) emulsifying the intermediate layer to obtain an emulsified fat mixture (also called nano-fat);
(5) centrifuging the emulsified fat mixture, thereby obtaining an intermediate liquid layer, i.e. a primary fat extract; and
(6) filtering and degerming the fat primary extract, thereby obtaining the cell-free fat extract.

14. A cell-free fat extract, wherein the cell-free fat extract is prepared by the method of claim 13.

15. (canceled)
